(19) United States
(12) Patent Application Publication
(10) Pub. No.: US 2012/0109244 A1
(43) Pub. Date: May 3, 2012

(54) PARAMETERS IN MONITORING CARDIAC RESYNCHRONIZATION THERAPY RESPONSE

(75) Inventors: Dave Anderson, Castaic, CA (US); Xiaoyi Min, Thousand Oaks, CA (US)

(73) Assignee: PACESETTER, INC., Sylmar, CA (US)

(21) Appl. No.: 12/938,579

(22) Filed: Nov. 3, 2010

(51) Int. Cl. A61N 1/365

(52) U.S. Cl. 607/17

(57) ABSTRACT

An exemplary method includes analyzing data from multiple parameters detected by an implantable cardiac device and determining an extent of heart failure (HF) progression. The parameters may include electrical synchrony, mechanical synchrony, and/or electromechanical delay (EMD). A change in a width of the native and/or paced QRS complex may provide a measure of electrical synchrony. Characterization of a delay between local cardiac impedance (CI) and global CI may provide a mechanical dyssynchrony index. A delay between the timing of a peak of the QRS complex and LV contraction (e.g., detected by SVC-CAN impedance) may provide a measure for EMD. Each of the parameters may be analyzed independently or collectively to assess HF progression. Based on the analysis, one or more pacing delays (e.g. AV/PV and/or VV) of the implantable cardiac device may be modified. Other exemplary methods, devices, systems, etc., are also disclosed.

### TECHNICAL FIELD

Exemplary technologies presented herein generally relate to cardiac pacing and/or stimulation therapy. Various techniques provide for monitoring heart failure progression and optimizing cardiac pacing therapy.

### BACKGROUND

Heart Failure (HF) is a chronic condition that affects over 5 million Americans and, according to the American Heart Association, HF accounts for more hospitalization among elderly people than any other condition. Heart failure is not a condition in which the heart abruptly stops beating. Instead, HF refers to a dysfunction in the pumping action of the heart due to the heart's inability to contract or relax properly. It is generally experienced by patients who have suffered a heart attack or whose hearts have been damaged by other conditions which have disrupted the heart's natural electrical conduction system.

The right ventricle of the heart is responsible for pumping blood to the lungs while the left ventricle is responsible for pumping blood to the rest of the body. The right atrium fills the right ventricle with deoxygenated blood while the left atrium fills the left ventricle with oxygenated blood. In a normal heart, the atria contract to fill the ventricles and then the ventricles contract in a synchronous manner to pump blood through the lungs or the body. Abnormal activation of any of the heart's four chambers reduces pumping efficiency. For example, abnormal atrial activation and contraction can decrease ventricular filling, cause abnormal ventricular wall motion, which may lead to mitral valve regurgitation (MR). Standard pharmacologic therapy cannot adequately resolve conduction and activation abnormalities such as left bundle branch block (LBBB) or a lengthy interventricular conduction delay (IVCD) that contribute to ventricular dyssynchrony.

Cardiac Resynchronization Therapy (CRT) provides an electrical solution to the symptoms and other difficulties brought on by HF. In many CRT systems, electrical impulses can be delivered to the tissue in the hearts two lower chambers (and typically one upper chamber). This is called biventricular pacing, and it causes the ventricles to beat in a more synchronized manner. Biventricular pacing improves the efficiency of each contraction of the heart and the amount of blood pumped to the body. This helps to lessen the symptoms of heart failure and, in many cases, helps to stop the progression of the disease. CRT can improve a variety of cardiac performance measures including cardiac index, decreased pulmonary artery pressures, decrease in myocardial oxygen consumption, decrease in dynamic mitral regurgitation, increase in global ejection fraction, decrease in NYHA classification symptoms, increased quality of life scores, increased distance covered during a 6-minute walk test, etc. Effects such as reverse remodeling may also be seen, for example, three to six months after initiating CRT. Patients that show such improvements are classified as CRT “responders”. However, for a variety of reasons, about 20-30% of all patients do not respond to CRT.

As described herein, various exemplary technologies allow a clinician, a system, etc., to monitor progression (including improvement) of HF and optimize a configuration of an implantable cardiac therapy device, which may increase the percentage of patients that respond to CRT.

### SUMMARY

A method includes determining various parameters from signals received by an implantable stimulation device. The implantable device may be configured for delivery of CRT, pacing therapy, as well as detection and storage of detected parameters. A patient's heart may improve or undergo reverse remodeling as a result of receiving CRT. This improvement may be indicated by changes in electrical and/or mechanical properties of the heart. Analysis of electrical and mechanical parameters may be used to decide if pacing delays require modification due to the changes in the heart. In some cases, electrical parameters (e.g., QRS width) may be the basis for modifying pacing therapy. In other cases, mechanical parameters (e.g., mechanical synchrony and hence, improved atrial or ventricular contraction) may be the basis for modifying pacing therapy. A balance between electrical parameters and mechanical parameters may also be used to modify pacing therapy. A modification may include adjusting an AV/PV delay and/or a VV pacing delay generated by the implantable stimulation device in order to optimize CRT.

In general, the various devices, methods, etc., described herein, and equivalents thereof, are suitable for use detecting a variety of cardiac parameters and modifying pacing therapies accordingly.

### BRIEF DESCRIPTION OF THE DRAWINGS

Features and advantages of the described implementations can be more readily understood by reference to the following description taken in conjunction with the accompanying drawings.

FIG. 1 is a simplified diagram illustrating an exemplary implantable stimulation device in electrical communication with various leads implanted into a patient's heart.

FIG. 2 is a functional block diagram of an exemplary implantable stimulation device illustrating basic elements that are configured to provide cardioversion, defibrillation, pacing stimulation and/or other tissue and/or nerve stimulation. The implantable stimulation device is further configured to sense information and administer stimulation pulses responsive to such information.

FIG. 3 is a diagram of an exemplary method for modifying a cardiac pacing therapy based upon changes in a width of a QRS complex.

FIG. 4 is a diagram of an exemplary method for modifying a cardiac pacing therapy based upon changes in a mechanical synchrony index.

FIG. 5 is a diagram of an exemplary method for modifying a cardiac pacing therapy based upon an electromechanical delay (EMD) of a patient's heart.

FIG. 6 is a diagram of an exemplary method for optimizing one or more pacing delays based on one or more of electrical synchrony, mechanical synchrony, and electromechanical delay (EMD).

### DETAILED DESCRIPTION

The following description includes the best mode presently contemplated for practicing the described implementations. This description is not to be taken in a limiting sense, but rather is made merely for the purpose of describing the general principles of the implementations. The scope of the described implementations should be ascertained with reference to the issued claims. In the description that follows, like numerals or reference designators generally reference like parts or elements.

### Overview

As described herein, an implantable device can deliver cardiac resynchronization therapy (CRT) according to one or more programmable parameters. Such parameters are typically programmed by a clinician using a programmer, which is a computing device configured to communicate with an implantable device. For example, a patient may be required to visit a clinic periodically for a procedure that involves a clinician placing a programmer's telemetric wand in proximity to the patient's implanted cardiac therapy device to thereby acquire information from the implantable device, instruct the implantable device to perform a test or tests and optionally set one or more parameters germane to how the implantable device functions. Alternatively, the implanted cardiac therapy device may be programmed remotely. Successful CRT may lead to reverse remodeling of a patient's heart such that electrical and/or mechanical conditions change and one or more of the programmed parameters become suboptimal. Various exemplary techniques described herein pertain to observing indicia related to CRT and optimization of CRT.

### Exemplary Stimulation Device

The techniques described below are intended to be implemented in connection with any stimulation device that is configured or configurable to stimulate nerves and/or stimulate and/or shock a patient's heart.

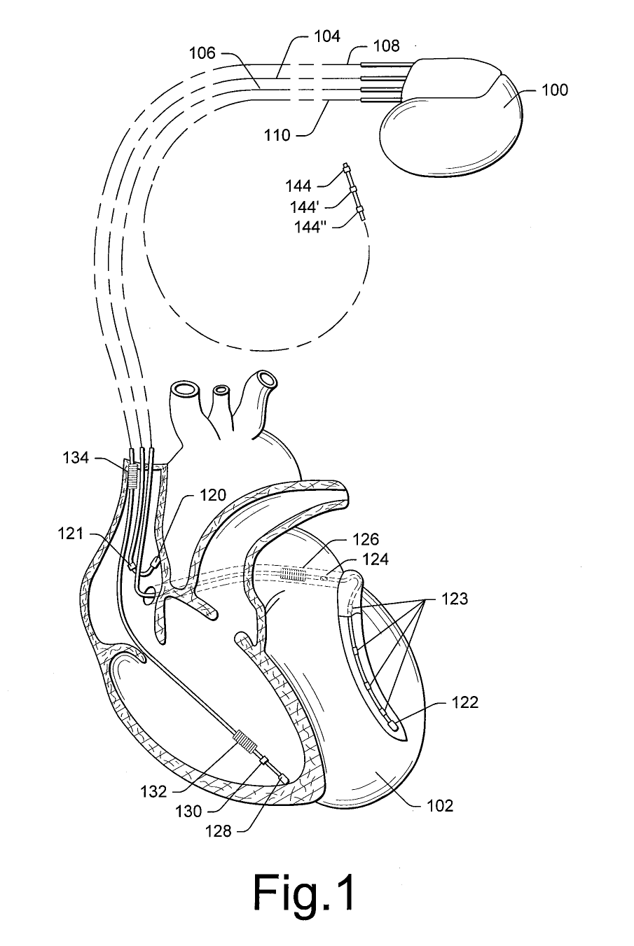

FIG. 1 shows an exemplary stimulation device 100 in electrical communication with a patient's heart 102 by way of three leads 104, 106, 108, suitable for delivering multi-chamber stimulation and shock therapy. The leads 104, 106, 108 are optionally configurable for delivery of stimulation pulses suitable for stimulation of autonomic nerves, non-myocardial tissue, other nerves, etc. In addition, the device 100 includes a fourth lead 110 having, in this implementation, three electrodes 144, 144′, 144″ suitable for stimulation of autonomic nerves, non-myocardial tissue, other nerves, etc. For example, this lead may be positioned in and/or near a patient's heart or near an autonomic nerve within a patient's body and remote from the heart.

The right atrial lead 104, as the name implies, is positioned in and/or passes through a patient's right atrium. The right atrial lead 104 optionally senses atrial cardiac signals and/or provide right atrial chamber stimulation therapy. As shown in FIG. 1, the stimulation device 100 is coupled to an implantable right atrial lead 104 having, for example, an atrial tip electrode 120, which typically is implanted in the patient's right atrial appendage. The lead 104, as shown in FIG. 1, also includes an atrial ring electrode 121. Of course, the lead 104 may have other electrodes as well. For example, the right atrial lead optionally includes a distal bifurcation having electrodes suitable for stimulation of autonomic nerves, non-myocardial tissue, other nerves, etc.

To sense atrial cardiac signals, ventricular cardiac signals and/or to provide chamber pacing therapy, particularly on the left side of a patient's heart, the stimulation device 100 is coupled to a coronary sinus lead 106 designed for placement in the coronary sinus and/or tributary veins of the coronary sinus. Thus, the coronary sinus lead 106 is optionally suitable for positioning at least one distal electrode adjacent to the left ventricle and/or additional electrode(s) adjacent to the left atrium. In a normal heart, tributary veins of the coronary sinus include, but may not be limited to, the great cardiac vein, the left marginal vein, the left posterior ventricular vein, the middle cardiac vein, and the small cardiac vein.

Accordingly, an exemplary coronary sinus lead 106 is optionally designed to receive atrial and ventricular cardiac signals and to deliver left ventricular pacing therapy using, for example, a series of electrodes 123 and/or a left ventricular tip electrode 122, left atrial pacing therapy using at least a left atrial ring electrode 124, and shocking therapy using at least a left atrial coil electrode 126. The series of electrodes 123 may be configured as a series of four electrodes shown here positioned in an anterior vein of the heart 102. For a complete description of a coronary sinus lead, the reader is directed to U.S. Pat. No. 5,466,254, “Coronary Sinus Lead with Atrial Sensing Capability” (Helland), which is incorporated herein by reference. The coronary sinus lead 106 further optionally includes electrodes for stimulation of autonomic nerves. Such a lead may include pacing and autonomic nerve stimulation functionality and may further include bifurcations or legs. For example, an exemplary coronary sinus lead includes pacing electrodes capable of delivering pacing pulses to a patient's left ventricle and at least one electrode capable of stimulating an autonomic nerve. An exemplary coronary sinus lead (or left ventricular lead or left atrial lead) may also include at least one electrode capable of stimulating an autonomic nerve, non-myocardial tissue, other nerves, etc., wherein such an electrode may be positioned on the lead or a bifurcation or leg of the lead.

Stimulation device 100 is also shown in electrical communication with the patient's heart 102 by way of an implantable right ventricular lead 108 having, in this exemplary implementation, a right ventricular tip electrode 128, a right ventricular ring electrode 130, a right ventricular (RV) coil electrode 132, and an SVC coil electrode 134. Typically, the right ventricular lead 108 is transvenously inserted into the heart 102 to place the right ventricular tip electrode 128 in the right ventricular apex so that the RV coil electrode 132 will be positioned in the right ventricle and the SVC coil electrode 134 will be positioned in the superior vena cava near the right atrium. Accordingly, the right ventricular lead 108 is capable of sensing or receiving cardiac signals, and delivering stimulation in the form of pacing and shock therapy to the right ventricle. An exemplary right ventricular lead may also include at least one electrode capable of stimulating an autonomic nerve, non-myocardial tissue, other nerves, etc., wherein such an electrode may be positioned on the lead or a bifurcation or leg of the lead.

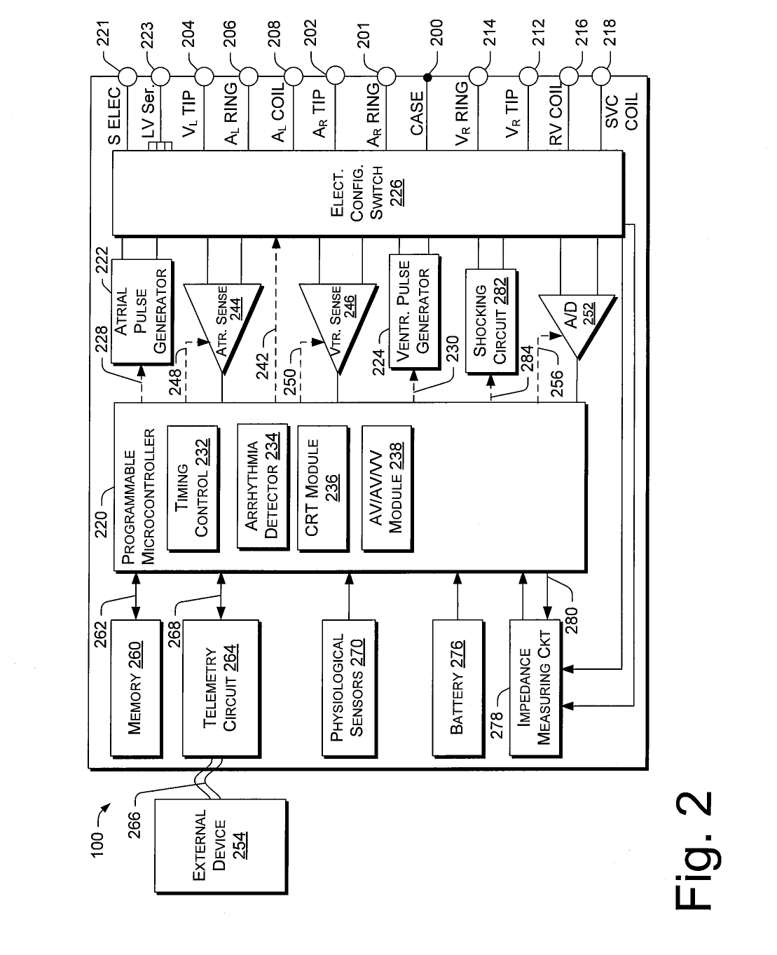

FIG. 2 shows an exemplary, simplified block diagram depicting various components of stimulation device 100. The stimulation device 100 can be capable of treating both fast and slow arrhythmias with stimulation therapy, including cardioversion, defibrillation, and pacing stimulation. The stimulation device can be solely or further capable of delivering stimuli to autonomic nerves, non-myocardial tissue, other nerves, etc. While a particular multi-chamber device is shown, it is to be appreciated and understood that this is done for illustration purposes only. Thus, the techniques and methods described below can be implemented in connection with any suitably configured or configurable stimulation device. Accordingly, one of skill in the art could readily duplicate, eliminate, or disable the appropriate circuitry in any desired combination to provide a device capable of treating the appropriate chamber(s) or regions of a patient's heart with cardioversion, defibrillation, pacing stimulation, autonomic nerve stimulation, non-myocardial tissue stimulation, other nerve stimulation, etc.

Housing 200 for stimulation device 100 is often referred to as the “can”, “case” or “case electrode”, and may be programmably selected to act as the return electrode for all “unipolar” modes. Housing 200 may further be used as a return electrode alone or in combination with one or more of the coil electrodes 126, 132, and 134 for shocking purposes. Housing 200 further includes a connector (not shown) having a plurality of terminals 201, 202, 204, 206, 208, 212, 214, 216, 218, 221, 223 (shown schematically and, for convenience, the names of the electrodes to which they are connected are shown next to the terminals).

To achieve right atrial sensing and/or pacing, the connector includes at least a right atrial tip terminal (A_{R} TIP) 202 adapted for connection to the atrial tip electrode 120. A right atrial ring terminal (A_{R} RING) 201 is also shown, which is adapted for connection to the atrial ring electrode 121. To achieve left chamber sensing, pacing and/or shocking, the connector includes at least a left ventricular tip terminal (V_{L} TIP) 204, a left atrial ring terminal (A_{L} RING) 206, and a left atrial shocking terminal (A_{L} COIL) 208, which are adapted for connection to the left ventricular tip electrode 122, the left atrial ring electrode 124, and the left atrial coil electrode 126, respectively. Connection to suitable autonomic nerve stimulation electrodes or other tissue stimulation or sensing electrodes is also possible via these and/or other terminals (e.g., via a nerve and/or tissue stimulation and/or sensing terminal S ELEC 221).

A terminal 223 allows for connection of a series of left ventricular electrodes. For example, the series of four electrodes 123 of the lead 106 may connect to the device 100 via the terminal 223. The terminal 223 and electrode configuration switch 226 allow for selection of one or more of the series of electrodes and hence electrode configuration. In the example of FIG. 2, the terminal 223 includes four branches to the switch 226 where each branch corresponds to one of the four electrodes 123.

To support right chamber sensing, pacing, and/or shocking, the connector further includes a right ventricular tip terminal (V_{R} TIP) 212, a right ventricular ring terminal (V_{R} RING) 214, a right ventricular shocking terminal (RV COIL) 216, and a superior vena cava shocking terminal (SVC COIL) 218, which are adapted for connection to the right ventricular tip electrode 128, right ventricular ring electrode 130, the RV coil electrode 132, and the SVC coil electrode 134, respectively. Connection to suitable autonomic nerve stimulation electrodes or other tissue stimulation or sensing electrodes is also possible via these and/or other terminals (e.g., via a nerve and/or tissue stimulation and/or sensing terminal S ELEC 221).

At the core of the stimulation device 100 is a programmable microcontroller 220 that controls the various modes of stimulation therapy. As is well known in the art, microcontroller 220 typically includes a microprocessor, or equivalent control circuitry, designed specifically for controlling the delivery of stimulation therapy, and may further include RAM or ROM memory, logic and timing circuitry, state machine circuitry, and I/O circuitry. Typically, microcontroller 220 includes the ability to process or monitor input signals (data or information) as controlled by a program code stored in a designated block of memory. The type of microcontroller is not critical to the described implementations. Rather, any suitable microcontroller 220 may be used that carries out the functions described herein. The use of microprocessor-based control circuits for performing timing and data analysis functions are well known in the art.

Representative types of control circuitry that may be used in connection with the described embodiments can include the microprocessor-based control system of U.S. Pat. No. 4,940,052 (Mann et al.), the state-machine of U.S. Pat. Nos. 4,712,555 (Thornander et al.) and 4,944,298 (Sholder), all of which are incorporated by reference herein. For a more detailed description of the various timing intervals used within the stimulation device and their inter-relationship, see U.S. Pat. No. 4,788,980 (Mann et al.), also incorporated herein by reference.

FIG. 2 also shows an atrial pulse generator 222 and a ventricular pulse generator 224 that generate pacing stimulation pulses for delivery by the right atrial lead 104, the coronary sinus lead 106, and/or the right ventricular lead 108 via an electrode configuration switch 226. It is understood that in order to provide stimulation therapy in each of the four chambers of the heart the atrial and ventricular pulse generators 222, 224 may include dedicated, independent pulse generators, multiplexed pulse generators, or shared pulse generators. The pulse generators 222, 224 are controlled by the microcontroller 220 via appropriate control signals 228, 230 respectively, to trigger or inhibit the stimulation pulses.

Microcontroller 220 further includes timing control circuitry 232 to control the timing of the stimulation pulses (e.g., pacing rate, atrio-ventricular delay (AV or more specifically AV_{RV} or AV_{LV}), atrial interconduction delay (e.g., A-A or more specifically A_{R}-A_{L} or A_{L}-A_{R}), or ventricular interconduction delay (VV or more specifically V_{LV}-V_{RV} or V_{RV}-V_{LV}), etc.) as well as to keep track of the timing of refractory periods, blanking intervals, noise detection windows, evoked response windows, alert intervals, marker channel timing, etc., which is well known in the art.

Microcontroller 220 further includes an arrhythmia detector 234. The detector 234 can be utilized by the stimulation device 100 for determining desirable times to administer various therapies. The detector 234 may be implemented in hardware as part of the microcontroller 220, or as software/firmware instructions programmed into the device and executed on the microcontroller 220 during certain modes of operation.

Microcontroller 220 further includes a cardiac resynchronization therapy (CRT) module 236 for performing a variety of tasks related to CRT. For example, the CRT module 236 may implement a therapy that relies on pacing a ventricle or pacing both ventricles to promote electrical synchrony, mechanical synchrony, and increased cardiac performance. The CRT module 236 may be implemented in hardware as part of the microcontroller 220, or as software/firmware instructions programmed into the device and executed on the microcontroller 220 during certain modes of operation. The CRT module 236 may optionally implement various exemplary methods described herein such as automatically revising pacing delays based on detected parameters. The CRT module 236 may also acquire cardiac information. Cardiac information may be in the form of signals, events or a combination of signals and events. For example, a detection algorithm may detect an atrial event and a ventricular event and note a time for each of these events. With respect to signals, the CRT module 236 may acquire electrograms that can be analyzed after their acquisition for any of a variety of features (e.g., a maximum slope as indicative of an evoked response, etc.). The electrograms may be stored in the memory 260.

Microcontroller 220 further includes an AA delay, AV delay and/or VV delay module 238 for performing a variety of tasks related to AA delay, AV delay and/or VV delay. This component can be utilized by the stimulation device 100 for determining desirable times to administer various therapies, including, but not limited to, ventricular stimulation therapy, bi-ventricular stimulation therapy, resynchronization therapy, atrial stimulation therapy, etc. This module 238 may include an optimization algorithm for automatically modifying the cardiac therapy timing parameters. The AA/AV/VV module 238 may be implemented in hardware as part of the microcontroller 220, or as software/firmware instructions programmed into the device and executed on the microcontroller 220 during certain modes of operation. Of course, such a module may be limited to one or more of the particular functions of AA delay, AV delay and/or VV delay. Such a module may include other capabilities related to other functions that may be germane to the delays.

The electronic configuration switch 226 includes a plurality of switches for connecting the desired electrodes to the appropriate I/O circuits, thereby providing complete electrode programmability. Accordingly, switch 226, in response to a control signal 242 from the microcontroller 220, determines the polarity of the stimulation pulses (e.g., unipolar, bipolar, combipolar, etc.) by selectively closing the appropriate combination of switches (not shown) as is known in the art.

Atrial sensing circuits 244 and ventricular sensing circuits 246 may also be selectively coupled to the right atrial lead 104, coronary sinus lead 106, and the right ventricular lead 108, through the switch 226 for detecting the presence of cardiac activity in each of the four chambers of the heart. Accordingly, the atrial and ventricular sensing circuits 244, 246 may include dedicated sense amplifiers, multiplexed amplifiers, or shared amplifiers. Switch 226 determines the “sensing polarity” of the cardiac signal by selectively closing the appropriate switches, as is also known in the art. In this way, the clinician may program the sensing polarity independent of the stimulation polarity. The sensing circuits (e.g., 244 and 246) are optionally capable of obtaining information indicative of tissue capture.

Each sensing circuit 244, 246 preferably employs one or more low power, precision amplifiers with programmable gain and/or automatic gain control, bandpass filtering, and a threshold detection circuit, as known in the art, to selectively sense the cardiac signal of interest. The automatic gain control enables the device 100 to deal effectively with the difficult problem of sensing the low amplitude signal characteristics of atrial or ventricular fibrillation.

The outputs of the atrial and ventricular sensing circuits 244, 246 are connected to the microcontroller 220, which, in turn, is able to trigger or inhibit the atrial and ventricular pulse generators 222, 224, respectively, in a demand fashion in response to the absence or presence of cardiac activity in the appropriate chambers of the heart. Furthermore, as described herein, the microcontroller 220 is also capable of analyzing information output from the sensing circuits 244, 246 and/or the data acquisition system 252 to determine or detect whether and to what degree tissue capture has occurred and to program a pulse, or pulses, in response to such determinations. The sensing circuits 244, 246, in turn, receive control signals over signal lines 248 and 250 from the microcontroller 220 for purposes of controlling the gain, threshold, polarization charge removal circuitry (not shown), and the timing of any blocking circuitry (not shown) coupled to the inputs of the sensing circuits 244, 246 as is known in the art.

For arrhythmia detection, the device 100 utilizes the atrial and ventricular sensing circuits 244, 246 to sense cardiac signals to determine whether a rhythm is physiologic or pathologic. In reference to arrhythmias, as used herein, “sensing” is reserved for the noting of an electrical signal or obtaining data (information), and “detection” is the processing (analysis) of these sensed signals and noting the presence of an arrhythmia. The timing intervals between sensed events (e.g., P-waves, R-waves, and depolarization signals associated with fibrillation) are then classified by the arrhythmia detector 234 of the microcontroller 220 by comparing them to a predefined rate zone limit (i.e., bradycardia, normal, low rate VT, high rate VT, and fibrillation rate zones) and various other characteristics (e.g., sudden onset, stability, physiologic sensors, and morphology, etc.) to determine a type of remedial therapy, if so desired (e.g., bradycardia pacing, anti-tachycardia pacing, cardioversion shocks or defibrillation shocks, collectively referred to as “tiered therapy”).

Cardiac signals are also applied to inputs of an analog-to-digital (A/D) data acquisition system 252. The data acquisition system 252 is configured to acquire intracardiac electrogram signals, convert the raw analog data into a digital signal, and store the digital signals for later processing and/or telemetric transmission to an external device 254. The data acquisition system 252 is coupled to the right atrial lead 104, the coronary sinus lead 106, the right ventricular lead 108 and/or the nerve or other tissue stimulation lead 110 through the switch 226 to sample cardiac signals across any pair of desired electrodes.

The microcontroller 220 is further coupled to a memory 260 by a suitable data/address bus 262, wherein the programmable operating parameters used by the microcontroller 220 are stored and modified, as required, in order to customize the operation of the stimulation device 100 to suit the needs of a particular patient. Such operating parameters define, for example, pacing pulse amplitude, pulse duration, electrode polarity, rate, sensitivity, automatic features, arrhythmia detection criteria, and the amplitude, waveshape, number of pulses, and vector of each shocking pulse to be delivered to the patient's heart 102 within each respective tier of therapy. One feature of the described embodiments is the ability to sense and store a relatively large amount of data (e.g., from the data acquisition system 252), which data may then be used for subsequent analysis to guide the programming of the device and optimized treatment.

Advantageously, the operating parameters of the implantable device 100 may be non-invasively programmed into the memory 260 through a telemetry circuit 264 in telemetric communication via communication link 266 with the external device 254, such as a programmer, transtelephonic transceiver, or a diagnostic system analyzer. The microcontroller 220 activates the telemetry circuit 264 with a control signal 268. The telemetry circuit 264 advantageously allows intracardiac electrograms (IEGMs) and status information relating to the operation of the device 100 (as contained in the microcontroller 220 or memory 260) to be sent to the external device 254 through an established communication link 266.

The stimulation device 100 can further include one or more physiologic sensors 270. For example, a physiologic sensor may be a “rate-responsive”sensor used to adjust pacing stimulation rate according to activity state of a patient. The one or more physiological sensors 270 may include a sensor to detect changes in cardiac output, changes in the physiological condition of the heart, or diurnal changes in activity (e.g., detecting sleep and wake states). Accordingly, the microcontroller 220 responds by adjusting the various pacing parameters (such as rate, AA delay, AV delay, VV delay, etc.) at which the atrial and ventricular pulse generators, 222 and 224, generate stimulation pulses.

While shown as being included within the stimulation device 100, it is to be understood that a physiologic sensor may also be external to the stimulation device 100, yet still be implanted within or carried by the patient. Examples of physiologic sensors that may be implemented in device 100 include known sensors that, for example, sense respiration rate, pH of blood, ventricular gradient, cardiac output, preload, afterload, contractility, hemodynamics, pressure, and so forth. Another sensor that may be used is one that detects activity variance, wherein an activity sensor is monitored diurnally to detect the low variance in the measurement corresponding to the sleep state. For a complete description of the activity variance sensor, the reader is directed to U.S. Pat. No. 5,476,483 (Bornzin et al.) which is hereby incorporated by reference.

The one or more physiological sensors 270 optionally include a minute ventilation sensor (e.g., where minute ventilation is defined as the total volume of air that moves in and out of a patient's lungs in a minute or tidal volume times number of breaths per minute). Signals generated by a sensor can be passed to the microcontroller 220 for analysis in determining whether to adjust the pacing rate, etc. In various configurations, the microcontroller 220 monitors signals for indications of activity status. Where a device includes a position sensor (e.g., accelerometer), the device may determine, for example, whether the patient is climbing upstairs or descending downstairs or whether the patient is sitting up after lying down.

The stimulation device additionally includes a battery 276 that provides operating power to all of the circuits shown in FIG. 2. For the stimulation device 100, which employs shocking therapy, the battery 276 is capable of operating at low current drains for long periods of time (e.g., preferably less than 10 μA), and is capable of providing high-current pulses (for capacitor charging) when the patient requires a shock pulse (e.g., preferably, in excess of 2 A, at voltages above 200 V, for periods of 10 seconds or more). The battery 276 also desirably has a predictable discharge characteristic so that elective replacement time can be detected.

The stimulation device 100 can further include magnet detection circuitry (not shown), coupled to the microcontroller 220, to detect when a magnet is placed over the stimulation device 100. A magnet may be used by a clinician to perform various test functions of the stimulation device 100 and/or to signal the microcontroller 220 that the external programmer 254 is in place to receive or transmit data to the microcontroller 220 through the telemetry circuits 264.

The stimulation device 100 further includes an impedance measuring circuit 278 that is enabled by the microcontroller 220 via a control signal 280. The known uses for an impedance measuring circuit 278 include, but are not limited to, lead impedance surveillance during the acute and chronic phases for proper lead positioning or dislodgement; detecting operable electrodes and automatically switching to an operable pair if dislodgement occurs; measuring respiration or minute ventilation; measuring thoracic impedance for determining shock thresholds; detecting when the device has been implanted; measuring stroke volume; and detecting the opening of heart valves, etc. The impedance measuring circuit 278 is advantageously coupled to the switch 226 so that any desired electrode may be used.

In the case where the stimulation device 100 is intended to operate as an implantable cardioverter/defibrillator (ICD) device, it detects the occurrence of an arrhythmia, and automatically applies an appropriate therapy to the heart aimed at terminating the detected arrhythmia. To this end, the microcontroller 220 further controls a shocking circuit 282 by way of a control signal 284. The shocking circuit 282 generates shocking pulses of low (e.g., up to 0.5 J), moderate (e.g., 0.5 J to 10 J), or high energy (e.g., 11 J to 40 J), as controlled by the microcontroller 220. Such shocking pulses are applied to the patient's heart 102 through at least two shocking electrodes, and as shown in this embodiment, selected from the left atrial coil electrode 126, the RV coil electrode 132, and/or the SVC coil electrode 134. As noted above, the housing 200 may act as an active electrode in combination with the RV electrode 132, or as part of a split electrical vector using the SVC coil electrode 134 or the left atrial coil electrode 126 (i.e., using the RV electrode as a common electrode).

Cardioversion level shocks are generally considered to be of low to moderate energy level (so as to minimize pain felt by the patient), and/or synchronized with an R-wave and/or pertaining to the treatment of tachycardia. Defibrillation shocks are generally of moderate to high energy level (i.e., corresponding to thresholds in the range of approximately 5 J to approximately 40 J), delivered asynchronously (since R-waves may be too disorganized), and pertaining exclusively to the treatment of ventricular fibrillation. Accordingly, the microcontroller 220 is capable of controlling the synchronous or asynchronous delivery of the shocking pulses.

As already mentioned, the implantable device 100 includes impedance measurement circuitry 278. Such a circuit may measure impedance or electrical resistance through use of various techniques. For example, the device 100 may deliver a low voltage (e.g., about 10 mV to about 20 mV) of alternating current between the RV tip electrode 128 and the case electrode 200. During delivery of this energy, the device 100 may measure resistance between these two electrodes where the resistance depends on any of a variety of factors. For example, the resistance may vary inversely with respect to volume of blood along the path.

In another example, resistance measurement occurs through use of a four terminal or electrode technique. For example, the exemplary device 100 may deliver an alternating current between one of the RV tip electrode 128 and the case electrode 200. During delivery, the device 100 may measure a potential between the RA ring electrode 121 and the RV ring electrode 130 where the potential is proportional to the resistance between the selected potential measurement electrodes.

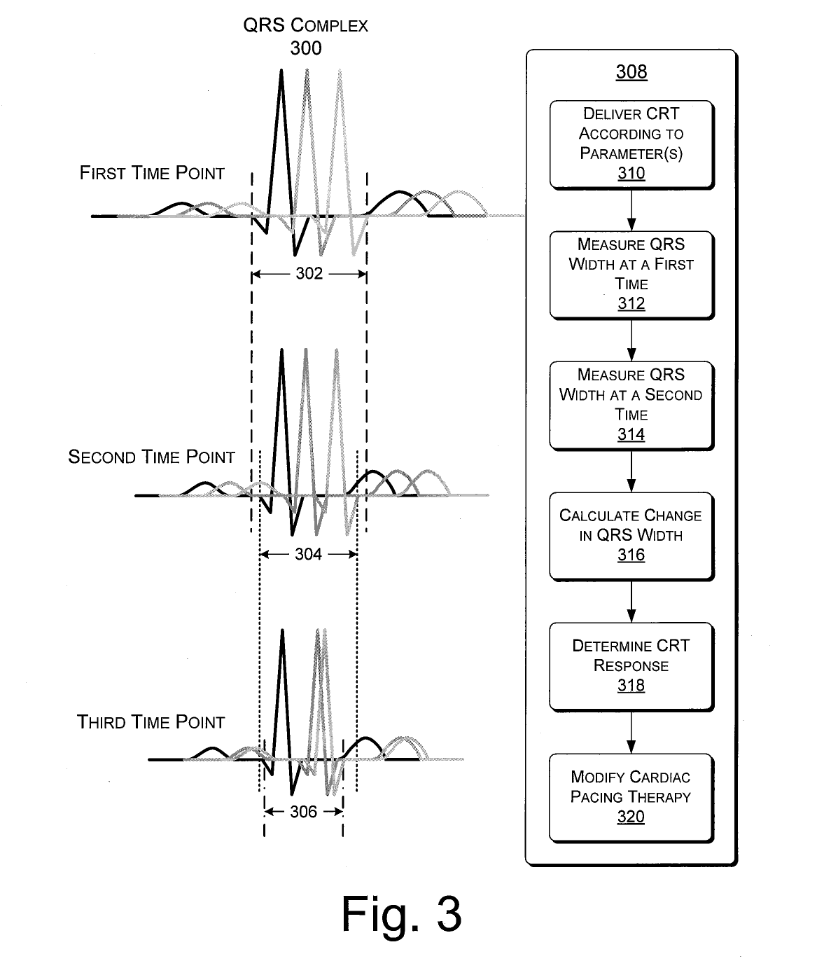

FIG. 3 shows the QRS complex 300 decreasing over time. Narrowing QRS width indicates better electrical synchrony and may also indicate that a patient is responding to CRT. However, baseline QRS, (i.e., before starting CRT) is questionable as a predictor for identifying CRT responders. Electrical activity such as the QRS complex may be measured using conventional techniques such as those for acquiring surface electrocardiograms or in vivo electrocardiograms (e.g., intracardiac electrograms). As described herein, the term “electrogram” (EGM) includes surface electrogram (ECG) and intracardiac electrogram (IEGM) as well as other types of electrograms that rely on one or more implanted electrodes. The IEGM may be sensed by the right ventricular lead 108 and the coronary sinus lead 106. Alternatively, IEGMs may be sensed between the SVC coil electrode 134 and the case or can of the sensing device 100. For additional description of techniques for sensing QRS using an implantable device, the reader is directed to U.S. Pat. No. 6,751,504 (Fishler), which is hereby incorporated by reference.

The QRS width may be measured between multiple vectors, such as, between any of electrodes connected to the right ventricular lead 108 and/or the coronary sinus lead 106. Each of the multiple vectors may use different electrodes to detect a QRS signal therefore each of the detected QRS signals may start and end at slightly different times. When shown together, the multiple QRS signals detected from multiple vectors may appear to be out of phase with each other because of the differing start and end times. Measuring the QRS width at multiple vectors rather than a single vector provides a more comprehensive overview of electrical activity of the heart. The width of the QRS signal when measured across multiple vectors is calculated from the earliest onset of QRS (e.g., at the vector of the first detected the QRS signal) to the last end of QRS (e.g. at the vector where the last QRS ends).

The exemplary QRS complex 300 decreases over time from a first width 302 to a second width 304 and to a third width 306. FIG. 3 shows the QRS complex 300 measured across three vectors resulting in three separate QRS signals. However, more or fewer than three vectors may also be used. Narrowing QRS width generally correlates with improved electrical synchrony and response to CRT. Conversely, a QRS width that remains unchanged or increases over time may indicate disease progression and/or lack of response to CRT. Each of the time points may be separated by several days, weeks, or months. The sampling times may correspond to clinical visits, for example, every three months.

The width of the QRS complex may be measured without pacing therapy (i.e., native width showing intrinsic conduction) or while receiving pacing at optimal delay settings. A QRS width measured by IEGM is determined by evaluating a combination of multiple vectors from intracardiac leads (e.g., right ventricular lead 108 and coronary sinus lead 106).

A method 308 shows one exemplary technique for modifying cardiac pacing therapy based at least in part upon a width of the QRS complex. In block 310, CRT is delivered to a patient according to one or more programmable parameters. The programmable parameters may include a width of the QRS complex. At block 312, a width of the QRS complex is measured at a first or initial time point. This may be a baseline QRS width determined shortly after implantation of an implantable device such as the device 100 of FIGS. 1 and 2. Following the initial measurement at block 314, a width of the QRS complex may be measured at a second, later time. As discussed above, the second time may be days, weeks, or months later than the first time. In some implementations, the measurements are performed automatically by the implantable device 100. An IEGM using the implanted electrodes may detect the QRS complex with either far-field sensing or near-field sensing as detected by the atrial and ventricular sensing circuits 244, 246 of the device 100. The programmable microcontroller 220 may interpret the signals sensed by the sensing circuits to measure a width of the QRS complex. Alternatively, an external processor such as in the external device 254 may receive raw data from the implantable device 100 and process the data to calculate a QRS width. The difference between a first QRS width and a second QRS width may be calculated at block 316 by the programmable microcontroller 220 or another processor. The memory 260 of the implantable device 100 may be used to store the QRS width data.

Given the calculated change in QRS width, at block 318, a determination is made about whether a patient is responding to CRT. The change in QRS width may be presented to a clinician for evaluation and he or she may characterize the extent of response or lack of response to CRT. Alternatively, block 318 may be implemented automatically by the microcontroller 222 of the device 100. For example, a narrowing of the QRS width may be characterized as indicating improved electrical synchrony, a constant QRS width may be interpreted by the microcontroller 222 as indicating no change in electrical synchrony, and an increase in the width of the QRS complex may indicate worsening electrical synchrony and possible non-responsiveness to CRT.

The method 308 may continue to block 320 at which cardiac pacing therapy is modified. In some implementations, the AV/PV delay and/or the VV delay may be modified to provide implantable device-based optimization of the QRS width. Such device-based optimization can allow for periodic adjustments to a patient's therapy (e.g., between clinic visits). Cardiac pacing therapy may also be modified by a clinician taking into account other factors of the patient's condition. The clinician-mediated modification may be performed during clinic visits every three months, six months, etc.

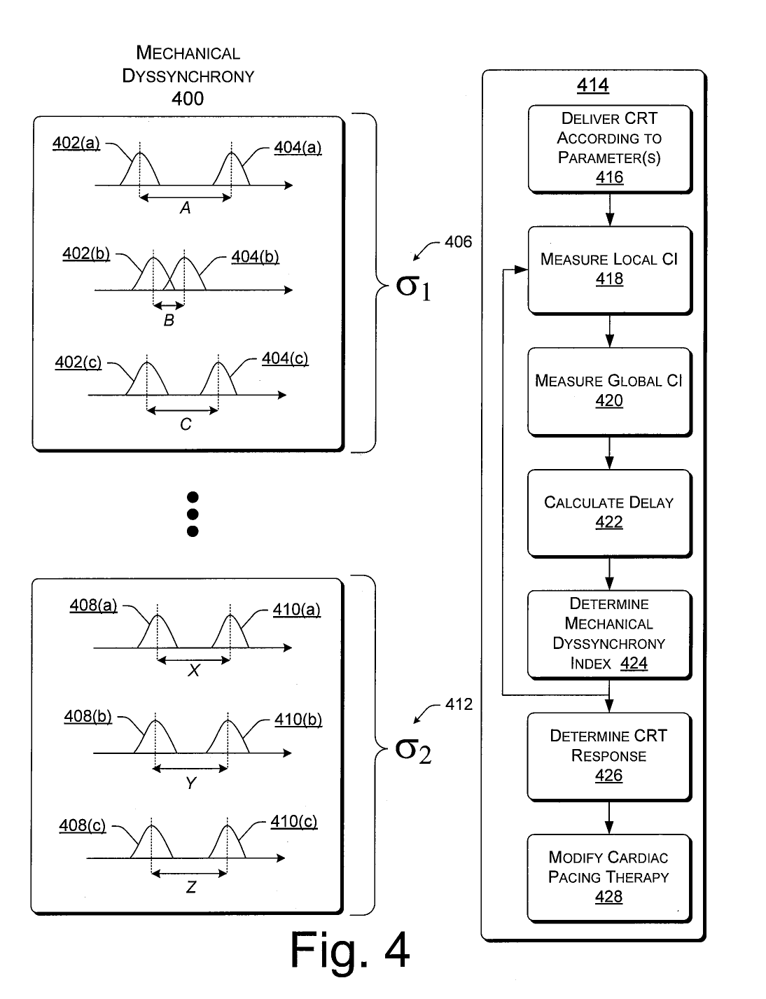

FIG. 4 shows a change in mechanical dyssynchrony 400 over time. Mechanical dyssynchrony between the LV and RV is associated with HF progression and has become acceptable as a predictive tool of hemodynamic and clinical response. However, assessment of mechanical dyssynchrony is commonly performed through TDI or 3D echo, which is costly and time-consuming. With the reliable sensing providing by an implantable device 100 that includes the components shown in FIGS. 1 and 2, it is possible to make a strong correlation between the cardiac impedances (CI) sensed over vectors between selected leads and mechanical dyssynchrony.

For example, a delay between a landmark associated with local CI and a landmark associated with a global CI provide indicia of mechanical dyssynchrony. Mechanical dyssynchrony can be assessed by the dispersion of timing contractions at each LV electrode and RV electrode. During a given observation window, a local CI landmark may be observed and recorded at multiple lead locations such as indicated by the center of impedance peaks 402(a), 402(b), and 402(c). For example, each of the electrodes 123 shown in FIG. 1 may provide information about timing of LV contraction. Similarly, a landmark for global CI 404(a), 404(b), and 404(c) follows the local CI landmarks 402 can also be measured at the same lead locations. In this example only three lead locations are represented, but any number of leads may be observed. The delays between the landmarks 402 and 404 for each of the respective leads are shown herein as A, B, and C. A mechanical dyssynchrony index may be derived from a standard deviation 406 of the delays A, B, and C. At a later time (e.g., three months later during a subsequent clinic visit) local CI and global CI may again be observed and recorded to identify any change in the dispersion of timing of contractions over the LV. For each lead observed at the later time, a landmark corresponding to local CI 408(a), 408(b), and 408(c) and a subsequent landmark corresponding to global CI 410(a), 410(b), and 410(c) may be observed and recorded. The respective delays X, Y, and Z between the landmarks for each lead are more uniform in this example resulting in a lower standard deviation 412 and a mechanical dyssynchrony index indicating improved mechanical synchrony or reverse remodeling in response to CRT.

Method 414 shows one exemplary technique for modifying cardiac pacing therapy based at least in part upon the mechanical dyssynchrony index. At block 416, CRT is delivered to a patient according to one or more programmable parameters. The programmable parameters may include a measure of mechanical dyssynchrony. At block 418, a local CI that indicates a timing of RV contraction is measured by the implantable device 100. The local CI may be measured by a variety of techniques including measuring impedance between the right ventricular lead 108 and coronary sinus lead 106, impedance at each LV electrode in a bipolar configuration, and the like. If a LV quadra-pole LV lead is used, CI at each LV electrode (e.g. bipolar configuration) is measured and used for local contraction onset assessment.

At block 420, global CI is measured. Global CI may be measured by the impedance between the SVC coil electrode 134 and the case or can of the sensing device 100. Max SVC-can impedance is associated with aortic valve opening and this may serve as one landmark for measuring mechanical dyssynchrony. Similarly, dZ/dt(max) may be used to time contraction. The landmarks may include the QRS sensed at the right ventricular lead 108, coronary sinus lead 106, or other leads.

A delay between the landmark associated with RV contraction and the landmark associated with LV contraction is calculated at block 422. Although a healthy heart may have synchronized RV and LV contractions, the specific landmarks selected may exhibit a delay even when the ventricles are perfectly synchronized.

Next at block 424 a mechanical dyssynchrony index is determined. As discussed above the mechanical dyssynchrony index may be the standard deviation of the delay between local CI and global Cl. The collection of impedance data and calculation of mechanical dyssynchrony across multiple cardiac cycles may be repeated at a later time to characterize HF progression. As discussed above, this later time may come days, weeks, or months later. Once at least two data points are collected, for example standard deviation 406 and standard deviation 412, response to CRT may be determined at block 426. Since the delay in the impedance peak (e.g., 404, 410) reflects the electromechanical delay, the dyssynchrony or more specifically the mechanical dyssynchrony index can be trended for purposes of monitoring and treating heart failure.

The method 414 may continue to block 428 at which cardiac pacing therapy is modified as discussed above with respect to block 318 of FIG. 3. Decreasing standard deviation over time is generally associated with improved mechanical synchrony whereas increasing or unchanged standard deviation may indicate lack of response to CRT. When considering CRT or no CRT, the mechanical dyssynchrony parameters can help decide if a patient is a responder, likely to be a responder or is not a responder.

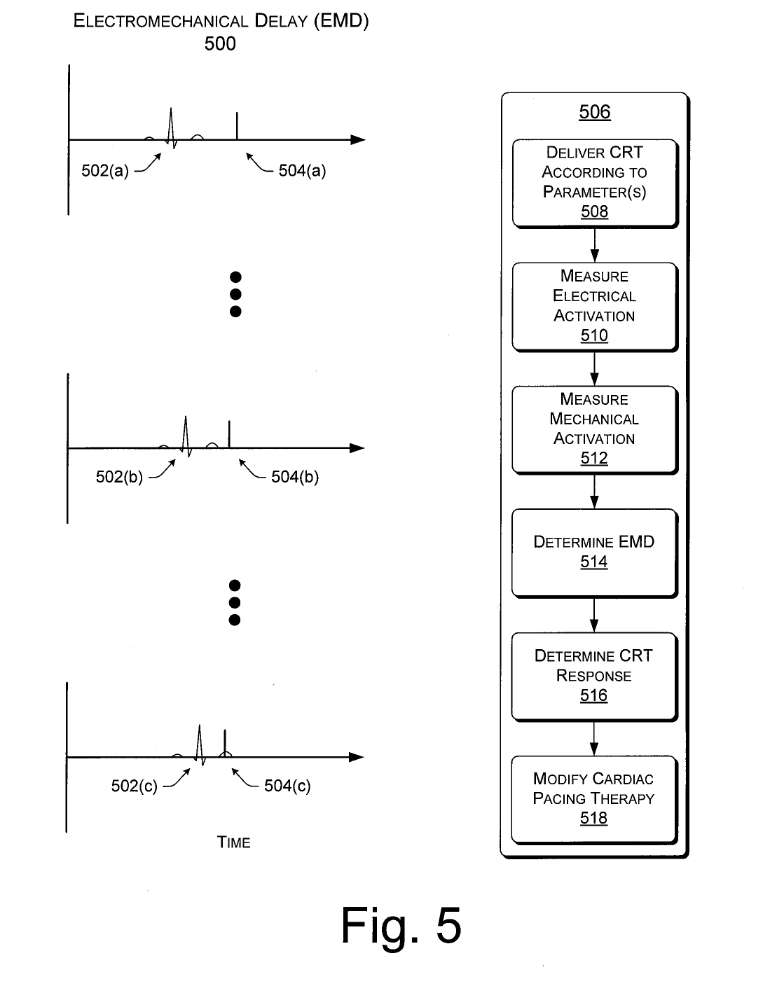

FIG. 5 shows an electromechanical delay (EMD) 500 between an electrical activation time 502(a) and a mechanical activation time 504(a). This delay is representative of the electromechanical delay of the respective ventricle. The EMD may be observed over time similar to the other parameters discussed above in order to detect changes that may indicate HF progression. In this example, a later measure of EMD shows a decreased time interval between an electrical activation time 502(b) and a mechanical activation time 504(b). This in turn is followed by a smaller delay between electrical activation 502(c) and mechanical activation 504(c) at a third observation time. In this example, the decreasing EMD indicates improved heart function and a patient exhibiting this change would likely be a responder to CRT.

Method 506 shows one exemplary technique for modifying cardiac pacing therapy based at least in part upon the electromechanical delay (EMD). At block 508, CRT is delivered to a patient according to one or more programmable parameters. The programmable parameters may include an EMD. At block 510, electrical activation is measured. The peak of a QRS complex may be used as an indicator of the time of electrical activation. At block 512, mechanical activation is measured. A time of LV contraction may represent the mechanical activation time. Leads of the implantable device 100 may determine the time of LV contraction based on impedance between the SVC coil electrode 134 and the case or can of the implantable device 100. The atrial and ventricular sensing circuits 244, 246 of the implantable device 100 may receive impedance signals that the programmable microcontroller 222 interprets as electrical activation and mechanical activation. An increase in the time delay between the occurrence of the QRS complex (available to the exemplary implantable device 100) and the occurrence of a corresponding peak in the impedance waveform can be indicative of worsening association between the electrical and mechanical activities of the corresponding ventricle.

Given the time of electrical activation and the time of mechanical activation, the electromechanical delay (EMD) may be determined at block 514. The programmable microcontroller 220 may calculate the delay by simply subtracting one time from the other. The implantable device 100 may also store the EMD data so that changes in EMD can be trended to determine whether a patient is responding to CRT at block 516.

The method 506 may continue to block 518 at which cardiac pacing therapy is modified as discussed above with respect to block 318 of FIG. 3 and block 428 of FIG. 4.

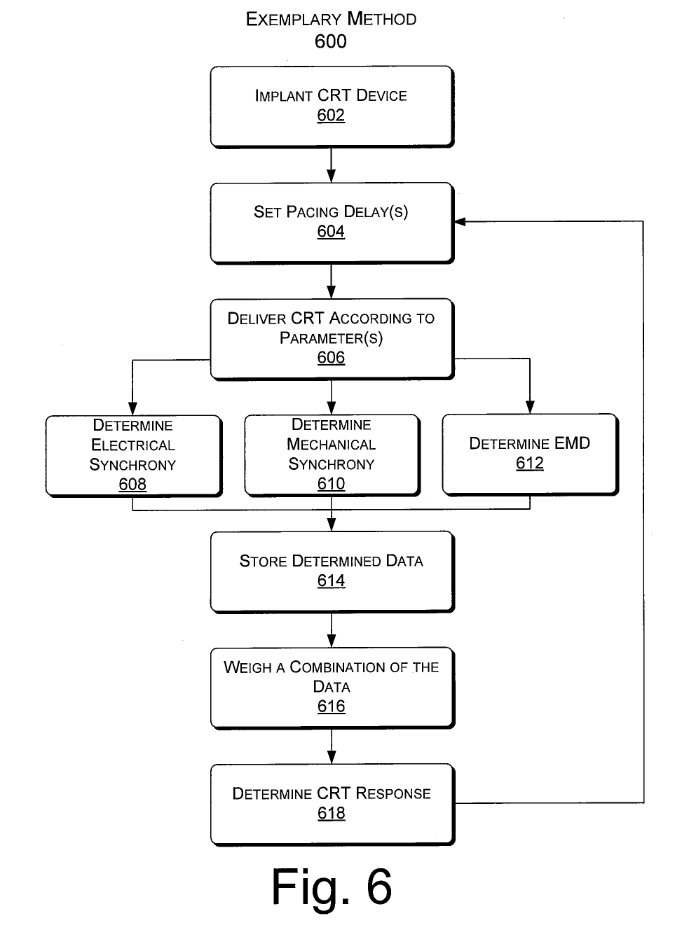

FIG. 6 shows a block diagram of an exemplary method 600 for monitoring HF and optimizing pacing delay settings. The method 600 commences at block 602 with implantation of a CRT device such as device 100 shown in FIGS. 1 and 2 in a patient. A clinician may initially set pacing delays at block 604 for one or more different electrode configurations (e.g. multisite pacing) or varying inter-stimulus timing (e.g. AV delay, VV delay) based on the condition of the patient's heart at the time of implantation. For example, an AV delay may be set at around 150 ms and the VV delay may range from 0-30 ms. Following the initial setting of pacing delays at block 604, the method 600 may proceed along any one or more of multiple paths in parallel. Once the initial pacing delays have been set at block 604, CRT is delivered to the patient according to one or more programmable parameters at block 606. The CRT may be delivered for a length of time such as days, weeks, or months.

At block 608, electrical synchrony of the patient's heart is determined as shown above in FIG. 3. Specifically, a change in the width of the QRS complex may be used as a measure of electrical synchrony. At block 610, mechanical synchrony is determined as shown in FIG. 4 above. Mechanical synchrony may be determined by calculating a delay between a landmark related to LV-RV impedance and a landmark related to SVC-CAN impedance. At block 612, an electromechanical delay (EMD) is determined by the method 506 shown above in FIG. 5. The period of EMD may be set as the time difference between the peak of the QRS complex and a timing of LV contraction.

At block 614, data collected by the implanted CRT device pertaining to electrical synchrony, mechanical synchrony, and/or electrical mechanical delay (EMD) may be stored either in the memory 260 of the device 100 and additionally or alternatively stored in a memory of an external device such as external device 254. Storing of data from different time points such as the multiple time points shown in FIG. 3, FIG. 4, and FIG. 5 above allows for trending of changes in mechanical and electrical heart function over extended periods of time such as days, weeks, or months.

At block 618, the response of the patient to CRT is determined either automatically by the device 100 or by a clinician. Depending on the changes in the observed parameters, the patient may be characterized as a responder or non-responder and treatment may be modified accordingly. One way of modifying treatment is adjusting the pacing delays and the method 600 returns to block 604 to accomplish this modification. Optimization of the pacing delays may be performed iteratively for example during every clinic visit or, in implementations in which the device 100 automatically optimizes delays, more frequently such as every week.

Optimization of delays can be done individually through each of parameters stated or a weighting of all the parameters into the consideration at block 616. Weighting may also remove one or more of the parameters from consideration by assigning a parameter a weight of zero. Specific weighting values may be determined experimentally, involve judgment of the clinician, and vary depending on the patient receiving treatment. For example, if the patient is not responding to CRT the clinician may shift emphasis from electrical synchrony to mechanical synchrony (e.g., increase the weight given to measures of mechanical synchrony while decreasing the weight given to measures of electrical synchrony). A balance between electrical synchrony and mechanical synchrony can be included in this approach. Instead of searching for modifications to only one of paced QRS width, or mechanical dyssynchrony index, or electrical-mechanical delays, a neutral point with defined improved mechanical synchrony and electrical synchrony can be used as optimal delay criteria.

The method 600 determines one or more synchrony and/or delay parameters. Subsequently, based on one or more of the parameters, optionally in conjunction with other information, a clinician or a device may select a configuration (e.g., AV/VV delays, etc.) that yielded or yields the best value(s) for the parameter(s). This configuration may then be used to modify the pacing delays at block 604.

With respect to measures and parameters used in optimization or delivery of a cardiac therapy (e.g., cardiac resynchronization therapy), these may include:
PP, AA Interval between successive atrial events
IACT Intra-atrial conduction time (see also ΔP, ΔA)
PV Delay between an atrial event and a paced ventricular event
PVₒₚₜᵢₘₐₗ Optimal PV delay
PV_{RV} PV delay for right ventricle
PV_{LV} PV delay for left ventricle
AV Delay for a paced atrial event and a paced ventricular event
AVₒₚₜᵢₘₐₗ Optimal AV delay
AV_{RV} AV delay for right ventricle
AV_{LV} AV delay for left ventricle
Δ Estimated interventricular delay (e.g., AV_{LV}−AV_{RV})
Δ_{programmed} Programmed interventricular delay (e.g., a programmed VV delay)
Δₒₚₜᵢₘₐₗ Optimal interventricular delay
IVCD_RL Delay between an RV event and a consequent sensed LV event
IVCD_LR Delay between an LV event and a consequent sensed RV event
Δ_{IVCD} Difference in interventricular conduction delays (IVCD_LR−IVCD_RL)
ΔP, ΔA Width of an atrial event

As described herein, various techniques can be used to optimize CRT. Such techniques may optionally include use of external measurement or sensing equipment (e.g., echocardiogram, etc.). Further, use of internal measurement or sensing equipment for sensing pressure or other indicators of hemodynamic performance is optional. Adjustment and learning may rely on IEGM information and/or cardiac other rhythm information. In general, the method 600 of FIG. 6 aims to ensure that electrical and mechanical parameters are used for continuing CRT optimization in patients that are receiving CRT.

### CONCLUSION

Although exemplary methods, devices, systems, etc., have been described in language specific to structural features and/or methodological acts, it is to be understood that the subject matter defined in the appended claims is not necessarily limited to the specific features or acts described. Rather, the specific features and acts are disclosed as exemplary forms of implementing the claimed methods, devices, systems, etc.

What is claimed is:

1. A method of monitoring response of a patient to cardiac resynchronization therapy (CRT) delivered by an implantable medical device, said method comprising:
delivering CRT to the patient according to one or more programmable parameters;
measuring a width of a depolarization event observed at a plurality of vectors at a first time and later at a second time, the width measured from the earliest onset of the depolarization event observed at a one of the plurality of vectors to the last end of the depolarization event observed at a one of the plurality of vectors; and
determining whether the patient is responding to CRT based at least in part on a change in the width of the depolarization event.

2. The method of claim 1, wherein the measuring the width of the depolarization event includes measuring a width of a QRS complex.

3. The method of claim 2, wherein the measuring the width of the QRS complex includes sensing the QRS complex using far-field sensing.

4. The method of claim 2, wherein the measuring the width of the QRS complex includes sensing the QRS complex using near-field sensing.

5. The method of claim 2, wherein the first QRS width and the second QRS width are intrinsic QRS widths measured in the absence of artificial pacing.

6. The method of claim 2, wherein the first QRS width and the second QRS width are measured during artificial pacing.

7. The method of claim 1, further comprising modifying one or more parameters for delivery of cardiac pacing therapy based at least in part on the change in the width of the depolarization event.

8. The method of claim 7, wherein the one or more parameters include one of an AV-delay value, a PV-delay value or a VV-delay value.

9. A method of monitoring response of a patient to cardiac resynchronization therapy (CRT) delivered by an implantable medical device, said method comprising:
delivering CRT to the patient according to one or more programmable parameters;
measuring a local cardiac impedance indicating a time of RV contraction and a global cardiac impedance indicating a time of LV contraction over multiple cardiac cycles;
calculating a delay between the time of RV contraction and the time of LV contraction for each of the cardiac cycles;
determining a mechanical dyssynchrony index based at least in part on the delay for each of the cardiac cycles;
repeating the measuring, calculating, and determining at a later time; and
determining whether the patient is responding to CRT based at least in part on a change in the mechanical dyssynchrony index.

10. The method of claim 9, wherein decreasing standard deviation associated with the mechanical dyssynchrony index indicates improved mechanical synchrony.

11. The method of claim 9, wherein measuring the local cardiac impedance includes measuring impedance between a LV lead and a RV lead of the implantable medical device.

12. The method of claim 9, wherein measuring the local cardiac impedance includes measuring impedance at a multiple bipolar LV leads.

13. The method of claim 9, wherein measuring the global cardiac impedance includes measuring impedance between a SVC lead of an implantable medical devices and a housing of the implantable medical device.

14. The method of claim 9, wherein the time of RV contraction is based at least in part on timing of a QRS complex sensed at a RV lead.

15. The method of claim 9, wherein the time of LV contraction is based at least in part on timing of a QRS complex sensed at a LV lead.

16. The method of claim 9, measuring the local cardiac impedance includes measuring impedance between a LV lead and a RV lead of the implantable medical device and measuring the global cardiac impedance includes measuring impedance between a SVC lead of the implantable medical device and a housing of the implantable medical device.

17. The method of claim 9, wherein calculating the delay includes calculating a delay between a time of maximum rate of change of the impedance between the LV lead and the RV lead and a time of maximum impedance between the SVC lead and the implantable medical device.

18. The method of claim 9, further comprising modifying one or more parameters for delivery of cardiac pacing therapy based at least in part on the change in the mechanical dyssynchrony index.

19. The method of claim 18, wherein the one or more parameters include one of an AV-delay value, a PV-delay value or a VV-delay value.

20. A method of monitoring response of a patient to cardiac resynchronization therapy (CRT) delivered by an implantable medical device, the method comprising:
delivering CRT to the patient according to one or more programmable parameters;
measuring an electrical activation time;
measuring a mechanical activation time comprising a time of LV contraction determined at least in part by an impedance between a SVC lead of an implantable medical device and a housing of the implantable medical device;
determining an electromechanical delay (EMD) based at least in part on a time interval between the electrical activation time and the mechanical activation time; and
determining whether the patient is responding to CRT based at least in part on the electromechanical delay (EMD).

21. The method of claim 20, wherein the electrical activation time comprises the time corresponding to a peak of a QRS complex.

22. The method of claim 20, further comprising modifying one or more parameters for delivery of cardiac pacing therapy based at least in part on the change in the electromechanical delay (EMD).

23. The method of claim 22, wherein the one or more parameters includes one of an AV-delay value, a PV-delay value or a VV-delay value.

24. A method of monitoring response of a patient to cardiac resynchronization therapy (CRT) delivered by an implantable medical device, the method comprising:
delivering CRT to the patient according to one or more programmable parameters;
determining a measure of electrical synchrony based at least in part on measuring a width of a QRS complex at a first time and later at a second time and detecting a change in the width of the QRS complex;
determining a measure of mechanical synchrony based at least in part on calculating a delay between a LV-RV impedance and an SVC-CAN impedance at a first time and later at a second time and detecting a change in the delay;
determining an electromechanical delay (EMD) based at least in part on a difference between a time corresponding to a peak of the QRS complex and a time corresponding to a LV contraction; and
determining whether the patient is responding to CRT based on the measure of electrical synchrony, the measure of mechanical synchrony, and the electromechanical delay (EMD).

25. The method of claim 24, further comprising storing the measure of electrical synchrony, the measure of mechanical synchrony, and the electromechanical delay (EMD) in the implantable medical device (IMD).

26. The method of claim 24, further comprising modifying one or more parameters for delivery of cardiac pacing therapy based at least in part on the measure of electrical synchrony, the measure of mechanical synchrony, and the electromechanical delay (EMD).

27. The method of claim 26, wherein the one or more parameters includes one of an AV-delay value, a PV-delay value or a VV-delay value.

28. The method of claim 26, wherein the modifying one or more parameters is based at least in part on a weighted combination of the measure of electrical synchrony, the measure of mechanical synchrony, and the electromechanical delay (EMD).
